(19) United States
(12) Patent Application Publication
(10) Pub. No.: US 2009/0214531 A1
(43) Pub. Date: Aug. 27, 2009

(54) Methods and Compositions for Treating and Preventing Bacterial Infections

(76) Inventors: John Mekalanos, Charlestown, MA (US); Stefan Pukatzki, Edmonton (CA); Joseph Mougous, Seattle, WA (US)

Correspondence Address: CLARK & ELBING LLP, 101 FEDERAL STREET, BOSTON, MA, 02110, US

(21) Appl. No.: 12/223,371

(22) PCT Filed: Jan. 29, 2007
(86) PCT No.: PCT/US07/02216

§ 371 Date: Feb. 12, 2009

Related U.S. Application Data
(60) Provisional application No. 60/763,239, filed on Jan. 30, 2006.

(51) Int. Cl. A61K 39/395; A61K 39/40; A61K 39/02; A61K 39/108; A61K 39/104; A61K 39/106; C12Q 1/00; G01N 33/53; C12N 1/21; C07K 16/00; C07K 16/12

(52) U.S. Cl. 424/133.1; 424/164.1; 424/169.1; 424/170.1; 424/234.1; 424/257.1; 424/259.1; 424/260.1; 424/261.1; 435/4; 435/7.1; 435/252.3; 435/252.34; 530/387.1; 530/387.3; 530/388.4

(57) ABSTRACT

The invention features methods and compositions for treating or preventing Gram-negative bacterial infections.

### BACKGROUND OF THE INVENTION

The invention relates to the prevention and treatment of bacterial infections.

The genus Pseudomonas contains more than 140 species, most of which are saprophytic. More than 25 species are associated with humans. Most pseudomonads known to cause disease in humans are associated with opportunistic infections. These include P. aeruginosa, P. fluorescens, P. putida, P. cepacia, P. stutzeri, P. maltophilia, and P. putrefaciens. Only two species, P. mallei and P. pseudomallei, produce specific human diseases: glanders and melioidosis. P. aeruginosa and P. maltophilia account for approximately 80, percent of pseudomonads recovered from clinical specimens. Because of the frequency with which it is involved in human disease, P. aeruginosa has received the most attention. It is a ubiquitous free-living bacterium and is found in most moist environments. Although seldom causing disease in healthy individuals, it is a major threat to hospitalized patients, particularly those with serious underlying diseases such as cancer and burns. The high mortality associated with these infections is due to a combination of weakened host defenses, bacterial resistance to antibiotics, and the production of extracellular bacterial enzymes and toxins. It is the most common pathogen isolated from patients who have been hospitalized longer than 1 week. It is a frequent cause of nosocomial infections such as pneumonia, urinary tract infections (UTIs), and bacteremia, and also afflicts cystic fibrosis patients. Pseudomonal infections are complicated and can be life threatening.

Methods and compositions that are useful for preventing and treating pseudomonal infections as well as infections of other Gram-negative bacteria are needed.

### SUMMARY OF THE INVENTION

In general, the invention features methods and compositions for treating or preventing Gram-negative bacterial infections.

Accordingly, in a first aspect, the invention features method of treating or preventing a Gram-negative bacterial infection in a patient by providing a humanized or human Hcp, VgrG, or Saf antibody or antibody fragment, and systemically administering the antibody to the patient.

In a related aspect, the invention features a method of treating or preventing Pseudomonas aeruginosa infection in a patient comprising the steps of providing a humanized or human Hcp, VgrG1, VgrG2, VgrG3, SafA, SafB, and SafC antibody or antibody fragment, and administering the antibody or fragment to the lungs of the patient.

The invention also features a purified antibody (or antibody fragment) specific for Hcp, VgrG, or Saf protein of a Gram-negative bacterium (e.g., Pseudomonas aeruginosa, Salmonella enterica, Escherichia coli, Vibrio cholerae, Yersinia enterocolitica, Legionella pneumophilia, Enterobacter aerogenes, Proteus morganii, Klebsiella pneumoniae, Burkholderia cepacia, Burkholderia pseudomallei, Shigella flexneri, or Shigella dysenteriae). The antibody may be a monoclonal antibody or a polyclonal antibody, and an antibody fragment can usefully be derived from either. Desirably, the antibody or fragment thereof is humanized or is human.

The invention also features a pharmaceutical composition that includes a purified antibody or antibody fragment specific for Hcp, VgrG, or Saf protein from a Gram-negative bacterium and a pharmaceutically acceptable carrier.

The invention features a method for treating or preventing a Gram-negative bacterial infection in a patient or reducing the pathogenicity of a Gram-negative bacterium by administering to the patient an effective amount of a pharmaceutical composition that includes a purified antibody or antibody fragment specific for Hcp, VgrG, or Saf protein from a Gram-negative bacterium and a pharmaceutically acceptable carrier.

The invention also features a method of inhibiting infection of a Gram-negative bacterium in a patient in need thereof by administering to the patient an effective amount of an Hcp, VgrG, or Saf antigen (i.e., an immunogenic Hcp, VgrG, or Saf polypeptide). If desired, the antigen can be a fragment of the Hcp protein that is capable of inducing an immune response. In one embodiment, the patient is inoculated with a gene vaccine having DNA encoding the Hcp antigen.

The invention also features a method of preventing or treating a Gram negative bacterial infection in a patient by administering an effective amount of a compound that inhibits Hcp or VgrG secretion or activity in the patient.

In another aspect, the invention features an attenuated bacterial mutant which contains a mutation of a gene of an IAHP locus. Desirably, the attenuated bacterial mutant exhibits 10× attenuation (i.e., LD50 values that are increased by 10-fold or more) in a standard animal model for chronic infection (see, e.g., Potvin et al., Environ. Microbiol. 5:1294-1308, 2003). The mutation can be an insertional inactivation or a gene deletion or substitution of one or more nucleotides of the gene, including, without limitation, of all nucleotides of the gene or of one, more than one or all nucleotides of a regulatory sequence of such a gene.

The invention also features several methods for identifying antimicrobial drugs. One such method includes the steps of: (a) contacting a candidate compound and a polypeptide encoded by a gene of an IAHP locus; and (b) comparing the biological activity of the polypeptide in the presence and absence of the candidate compound, wherein alteration of the biological activity of the polypeptide indicates that the candidate compound is an antimicrobial drug. Such alteration may be an increase or decrease in a biological activity exhibited by the polypeptide in the absence of the candidate compound or, alternatively, may be performance of a new and/or different biological activity by the polypeptide.

In another method for identifying an antimicrobial drug, a candidate compound is contacted with a polypeptide encoded by a gene of an IAHP locus; and binding of the candidate compound and the polypeptide is detected. Binding indicates that the candidate compound is an antimicrobial drug.

Another method for identifying an antimicrobial drug includes the steps of: (a) contacting a candidate compound and a Gram negative bacterium; and (b) detecting secretion of Hcp or VgrG. A decrease in Hcp or VgrG secretion, relative to secretion by the Gram negative bacterium not contacted with the candidate compound, indicates that the candidate compound is an antimicrobial drug.

In any of the foregoing aspects, the Gram-negative bacterium preferably is Pseudomonas aeruginosa, Salmonella enterica, Escherichia coli, Vibrio cholerae, Yersinia enterocolitica, Legionella pneumophilia, Enterobacter aerogenes, Proteus morganii, Klebsiella pneumoniae, Burkholderia cepacia, Burkholderia pseudomallei, Shigella flexneri, or Shigella dysenteriae.

Any patient having a Gram-negative bacterial infection (or at risk for having one) may be treated with the methods and compositions of the invention, including burn patients, surgical patients, prosthesis recipients, respiratory patients, cancer patients, cystic fibrosis patients, and immunocompromised patients.

### BRIEF DESCRIPTION OF THE DRAWINGS

FIG. 1. V. cholerae cytotoxicity toward the simple eukaryote D. discoideum. (A) Plaque assay. D. discoideum cells were plated on SM/5 with K. aerogenes and V. cholerae strains N16961, V52, SP120 (V52ΔvasK), and SP219 (V52ΔhlyA) at a density of 100 amoebae per plate. Bacterial virulence potential was determined by the number of plaques formed by D. discoideum in bacterial lawns. (B) Hemolytic phenotype of K. aerogenes and V. cholerae strains N16961, V52, SP120, and SP219 on trypticase soy agar containing 5% sheep blood. (C) Killing assay. Virulence of indicated bacteria was determined by enumerating the number of live amoebae recovered from bacterial lawns after a 24-h incubation. Numbers above the columns indicate fold change of number of amoebae in bacterial lawns over a 24-h period. Results shown are the means (±SD) of triplicate determinations.

FIG. 2. Genetic organization of the VAS pathway of V. cholerae. Horizontal gray arrows designate hypothetical genes, black arrows designate genes with homologues of known function, and empty arrows indicate genes of known function in V. cholerae (drawn to scale). Vertical arrows indicate transposon insertion sites in Dictyostelium-attenuated V. cholerae mutants.

FIG. 3. VAS-dependent secretion. (A) Secretion profiles of V. cholerae VAS mutants. SDS/PAGE of concentrated midlog culture supernatants of indicated strains. Black arrow indicates position of Hcp. (B) Extracellular secretion of epitope-tagged substrates. V. cholerae strains V52 and SP120 V52ΔvasK) maintaining a plasmid that allows arabinose-induced expression of tagged Hcp-2 and VgrG-2 were grown under inducing conditions. Cells and filtered supernatants were left untreated or incubated with either 0.1 mg/ml proteinase K (P.K) in the presence or absence of 1% SDS. Protease inhibitor PMSF was used to stop proteolysis after 20 min, and extracts were separated on a SDS/PAGE for immunoblotting with vesicular stomatitis virus glycoprotein antisera. The quality of pellet and supernatant fractionation was determined by localizing periplasmic β-lactamase (bla).

FIG. 4. V. cholerae cytotoxicity toward J774 macrophages. J774 cells were infected for 2 h with V52 (wild type) and isogenic mutant SP500 (ΔrtxA, ΔhlyA) or mutant SP501 (ΔrtxA, ΔhlyA, ΔvasK). Cells were fixed with 3% formaldehyde to assess the morphology of infected cells.

FIG. 5. Schematic representation of the three P. aeruginosa IAHP loci. IAHP open reading frames (ORFs) not discussed in the text (white) and non-IAHP ORFs that lie within the predicted IAHP operons (black) are labeled with their genome annotation ORF number. Grey ORFs are discussed in the text and are labeled according to their gene name or their closest homolog outside of other IAHP loci. Predicted orthologous ORFs with prior characterization and those characterized in this study are colored consistently in each locus. The boxed insert shows the position of the hcp2/vgrG2 locus encoded elsewhere on the genome.

FIG. 6. RetS regulates IAHP-I-dependent secretion of Hcp1. (A) SDS-PAGE analysis of concentrated culture supernatants from various P. aeruginosa strains. No differences are apparent between wild-type (PA01) and ΔclpB1-3*. The arrow highlights the position of secreted Hcp1 in ΔretS. This band is lacking from ΔretS Δhcp1. (B) Western blot analysis of IAHP-I-dependent secretion of Hcp1-V. In addition to the genetic alterations indicated, each strain contains a C-terminal chromosomal fusion of hcp1 to a DNA sequence encoding the VSV-G epitope (hcp1-V). Equal quantities of cell (C) and supernatant (S) fractions were probed with antibodies specific for the α-subunit of RNA polymerase (RNAP) and the VSV-G epitope.

FIG. 7. ClpB1* is highly similar to ClpB, but is required for Hcp1 secretion and not for thermotolerance. (A) Comparison of the domain organization of E. coli ClpB and that predicted for P. aeruginosa ClpB 1*. The ClpB domain boundaries and functions are based on their prior assignment from structural studies of the protein. N- and C-terminal domains are shown in yellow, nucleotide-binding domains (NBD) in red and the ClpB/Hsp104-specific linker in green. The narrow region of the linker domain represents an extended coiled-coil. The proteins are 35% identical overall and 45% identical within the NB and linker domains. (B) Thermotolerance assay of P. aeruginosa strains bearing clpB or clpB* deletions. Cells were exposed to a 25 minute heat pulse at 55° C. and viability was determined by colony forming units.

FIG. 8. ClpB1* localizes to discrete foci in a IcmF1- and Hcp1-dependent manner. (A) Left-Western blot analysis of Hcp1-V in ΔretS clpB1*-gfp. Right-Western blot analysis of GFP in ΔretS clpB1*gfp. (B) Flow cytometry analysis comparing GFP expression in ΔretS clpB1*gfp (grey fill) to isogenic strains bearing additional mutations in icmF1 (yellow) and hcp1 (blue). clpB1*-gfp in the wild-type background is shown in green. Wild-type (black) and wild-type containing a plasmid expressing GFP (red) are included as controls. (C) Fluorescence microscopy of clpB1*-gfp strains in (B). TMA-DPH is a membrane dye used to highlight the outline of the cells.

FIG. 9. Hcp1 forms a hexameric ring with a large internal diameter. (A) Ribbon representation of the Hcp1 monomer colored by secondary structure: β-strands, red; α-helices, blue; and loops, green. Secondary element assignments used in the text are indicated. (B) Top-view of a ribbon representation of the crystallographic Hcp1 hexamer. The individual subunits are colored differently to highlight their organization. (C) Edge-on view of the Hcp1 hexamer shown in (B). (D) Region of the Hcp1 crystal lattice illustrating the packing of Hcp1 hexamers into tube structures. The conserved glycine residues of the strap are rendered as molecular surfaces to emphasize their position at the ring interfaces. (E) Gel filtration chromatograph of purified Hcp1. An arrow indicates the position where sample was removed for use in (F). (F) Electron microscopy and single particle analysis of Hcp1. Electron micrograph of Hcp1 negatively stained with 0.75% (w/v) uranyl formate. Scale bar is 100 nm. Left inset frames show representative class averages and right inset frames show the same averages after six-fold symmetrization. Inset scale bar is 10 nm.

FIG. 10. Conservation and electrostatic analysis of Hcp1 surface residues. (A) The outer circumference of Hcp1 is polar. Calculated vacuum electrostatic surface potential of Hcp1. Blue and red regions represent positive and negative potential, respectively. (B) The two faces, but not the inner or outer circumferences of Hcp1 are conserved. Sequence conservation of Hcp1 was calculated from an alignment of 107 Hcp proteins in 43 Gram-negative bacteria. The relative degree of conservation at each amino acid on the surface of Hcp1 is indicated by color, where red residues are highly conserved and white residues are poorly conserved.

### DETAILED DESCRIPTION OF THE INVENTION

The secretion of proteins is a common mechanism by which bacterial pathogens mediate interactions with their hosts. We report here the identification of Hcp1 as a novel secreted protein of Pseudomonas aeruginosa. Our data indicate that Hcp1 secretion is dependent on a cluster of conserved genes that have been implicated in the virulence of P. aeruginosa and other Gram-negative pathogens. Furthermore, we show that Hcp1 secretion in P. aeruginosa is coordinately regulated with known virulence determinants such as type III secretion and biofilm formation. Structural modeling and genetic analyses implicate a AAA+ family ATPase as being required for Hcp1 secretion. A fluorescent fusion to this protein localizes to discrete foci within the cell in a manner dependent on Hcp1 secretion, thereby providing evidence of an Hcp1 secretory apparatus. We also solved the X-ray crystal structure of Hcp1 and found that the protein forms a hexameric ring with a large internal diameter. Our analysis of the structure suggests that Hcp1 forms interactions on both its faces and is likely to facilitate the transport of a macromolecule. Due to the conservation of hcp and its associated secretory gene cluster, its function may be relevant to the pathogenesis of many bacteria.

### Therapy

The invention features method of treating or preventing a Gram-negative bacterial infection in a patient by obtaining an Hcp antibody or antibody fragment, and systemically administering the antibody to the patient. Desirably, the Hcp antibody is humanized or human.

In order to generate antibodies with improved performance and/or reduced antigenicity in the methods of the invention, the non-complementarity-determining regions (CDRs) of an antibody may be replaced with similar regions of conspecific or heterospecific antibodies while retaining the epitopic specificity of the original antibody. This is most clearly manifested in the development and use of “humanized” antibodies, in which non-human CDRs are covalently joined to human framework regions (FRs) and/or constant (Fc/pFc′) regions to produce a functional antibody. Methods for making and using such humanized antibodies are well-established and include recombinant DNA techniques known in the art. For example, a gene encoding the Fc constant region of a murine (or other species) monoclonal antibody molecule may be digested with restriction enzymes to remove the region encoding the murine Fc, and the equivalent portion of a gene encoding a human Fc constant region may be substituted. See, e.g., U.S. Pat. Nos. 6,984,720 and 4,816,567; U.S. Patent Application Publication 2006-0015952; and International Patent Publication Nos. WO87/02671 and WO86/01533.

Antibodies may be further humanized by replacing sequences of the Fv variable region which are not directly involved in antigen binding with equivalent sequences from human Fv variable regions. Useful methods for making such antibodies include isolating, manipulating, and expressing the nucleic acid sequences that encode all or part of immunoglobulin Fv variable regions from at least one of a heavy or light chain. Sources of such nucleic acid are well-known to those skilled in the art; for example, nucleic acid may be obtained from 7E3, an anti-GPII_{b}IIIₐ antibody producing hybridoma. The recombinant DNA encoding the chimeric antibody, or fragment thereof, can then be cloned into an appropriate expression vector. Suitable humanized antibodies can alternatively be produced by CDR substitution, as described, e.g., in U.S. Pat. No. 5,225,539; Jones et al., Nature 321:552-525, 1986; Verhoeyan et al., Science 239:1534, 1988; and Beidler, J. Immunol. 141:4053-4060, 1988. In addition, general reviews of humanized antibodies are provided by Morrison, Science 229:1202-1207, 1985, and Oi, BioTechniques 4:214, 1986.

As we have detected Hcp protein in the sputum of cystic fibrosis (CF) patients and Hcp antibodies in the sera of these patients, it is especially desirable to administer Hcp antibodies, or fragments thereof, to CF patients having an Pseudomonas aeruginosa infection. An antibody or antibody fragment may be administered systemically or, alternatively, may be administered directly to the lungs of the patient.

The invention features a method for treating or preventing a Gram-negative bacterial infection in a patient or reducing the pathogenicity of a Gram-negative bacterium by administering to the patient an effective amount of a pharmaceutical composition that includes a purified antibody or antibody fragment specific for Hcp protein of a Gram-negative bacterium and a pharmaceutically acceptable carrier.

The invention also features a method of inhibiting infection of a Gram-negative bacterium in a patient in need thereof by administering to the patient an effective amount of an Hcp antigen (i.e., an immunogenic Hcp polypeptide). If desired, the antigen can be a fragment of the Hcp protein that is capable of inducing an immune response. In one embodiment, the patient is inoculated with a gene vaccine having DNA encoding the Hcp antigen.

The invention also features a method of preventing or treating a Gram negative bacterial infection in a patient by administering an effective amount of a compound that inhibits Hcp in the patient. Such compounds include antibodies (as described herein) and small molecule inhibitors that may be identified, inter alia, using the screening methods described below.

### Antibodies

The invention features a purified antibody (or antibody fragment) specific for Hcp protein from a Gram-negative bacterium (e.g., Pseudomonas aeruginosa, Salmonella enterica, Escherichia coli, Vibrio cholerae, Yersinia enterocolitica, Legionella pneumophilia, Enterobacter aerogenes, Proteus morganii, Klebsiella pneumoniae, Burkholderia cepacia, Burkholderia pseudomallei, Shigella flexneri, or Shigella dysenteriae). The antibody may be a monoclonal antibody or a polyclonal antibody. Desirably, the antibody or fragment thereof is humanized or is human (see above). Methods for making antibodies are well known in the art.

The invention also features a pharmaceutical composition that includes a purified antibody or antibody fragment specific for Hcp protein from a Gram-negative bacterium and a pharmaceutically acceptable carrier. Suitable carriers are also well known in the art.

### Attenuated Bacterial Strains

The invention features an attenuated bacterial mutant which contains a mutation of a gene of an IAHP locus The mutation can be an insertion, deletion or substitution of one or more nucleotides of the gene and/or a regulatory sequence of such a gene, such that the amino acid sequence of the encoded gene is altered or the protein is not made, resulting in an increase, decrease or other alteration in biological activity of a protein encoded by such gene. Methods for making such mutants are described below.

### Methods for Identifying Antimicrobial Drugs

The invention features several methods for identifying antimicrobial drugs. One such method includes the steps of: (a) contacting a candidate compound with a polypeptide encoded by a gene of an IAHP locus; and (b) comparing the biological activity of the polypeptide in the presence and absence of the candidate compound, wherein alteration of the biological activity of the polypeptide indicates that the candidate compound is an antimicrobial drug.

In another method for identifying an antimicrobial drug, a candidate compound is contacted with a polypeptide encoded by a gene of an IAHP locus; and binding of the candidate compound and the polypeptide is detected. Binding indicates that the candidate compound is an antimicrobial drug.

Another method for identifying an antimicrobial drug includes the steps of: (a) contacting a candidate compound and a Gram negative bacterium; and (b) detecting secretion of Hcp. A statistically significant decrease in Hcp secretion, relative to secretion by the Gram negative bacterium not contacted with the candidate compound, indicates that the candidate compound is an antimicrobial drug.

Any screening assays known in the art, e.g., binding assays or displacement assays, may be used in the methods of the invention to measure compound-protein interactions in a screening strategy. Useful screening assays may employ, e.g., fluorescence polarization, mass spectrometry (Nelson and Krone, J. Mol. Recognit., 12:77-93, 1999), surface plasmon resonance (Spiga et al., FEBS Lett., 511:33-35, 2002; Rich and Mizka, J. Mol. Recognit., 14:223-8, 2001; Abrantes et al., Anal. Chem., 73:2828-35, 2001), fluorescence resonance energy transfer (FRET) (Bader et al., J. Biomol. Screen, 6:255-64, 2001; Song et al., Anal. Biochem. 291:133-41, 2001; Brockhoff et al., Cytometry, 44:338-48, 2001), bioluminescence resonance energy transfer (BRET) (Angers et al., Proc. Natl. Acad. Sci. USA, 97:3684-9, 2000; Xu et al., Proc. Natl. Acad. Sci. USA, 96:151-6, 1999), fluorescence quenching (Engelborghs, Spectrochim. Acta A. Mol. Biomol. Spectrosc., 57:2255-70, 70; Geoghegan et al., Bioconjug. Chem. 11:71-7, 2000), fluorescence activated cell scanning/sorting (Barth et al., J. Mol. Biol., 301:751-7, 2000), ELISA, or radioimmunoassay (RIA). Alternate methods for measuring compound-protein interactions are known to the skilled artisan.

Screening techniques useful in the methods of the invention may measure compound binding directly; alternatively, indirect readouts may be used. In one exemplary method for assaying compound binding to a protein involved in Hcp secretion, cells capable of secreting Hcp are cultured in the presence and absence of the test compound, and the level of Hcp secretion in each case is determined. Exposure of cells to a compound that binds to or otherwise inhibits a protein involved in the Hcp secretion pathway may result in a decrease in Hcp secretion relative to non-exposed cells, providing a readout of compound-protein binding.

In general, compounds useful for screening in the methods of the invention can be identified from a variety of sources, e.g., large libraries of natural products, synthetic (or semi-synthetic) extracts, or chemical libraries, according to methods known in the art.

### EXAMPLE 1

The bacterium Vibrio cholerae, like other human pathogens that reside in environmental reservoirs, survives predation by unicellular eukaryotes. Strains of the O1 and O139 serogroups cause cholera, whereas non-O1/non-O139 strains cause human infections through poorly defined mechanisms. Using Dictyostelium discoideum as a model host, we have identified a virulence mechanism in a non-O1/non-O139 V. cholerae strain that involves extracellular translocation of proteins that lack N-terminal hydrophobic leader sequences. Accordingly, we have named these genes “VAS” genes for virulence-associated secretion, and we propose that these genes encode a prototypic “type VI” secretion system. We show that vas genes are required for cytotoxicity of V. cholerae cells toward Dictyostelium amoebae and mammalian J774 macrophages by a contact-dependent mechanism. A large number of Gram-negative bacterial pathogens carry genes homologous to vas genes and potential effector proteins secreted by this pathway (i.e., hemolysin-coregulated protein and VgrG). Mutations in vas homologs in other bacterial species have been reported to attenuate virulence in animals and cultured macrophages. Thus, the genes encoding the VAS-related, type VI secretion system likely play an important conserved function in microbial pathogenesis and represent an additional class of targets for vaccine and antimicrobial drug-based therapies. Our findings are discussed in greater detail below.

The Non-O1, Non-O139 V. cholerae Strain V52 Resists D. discoideum Predation

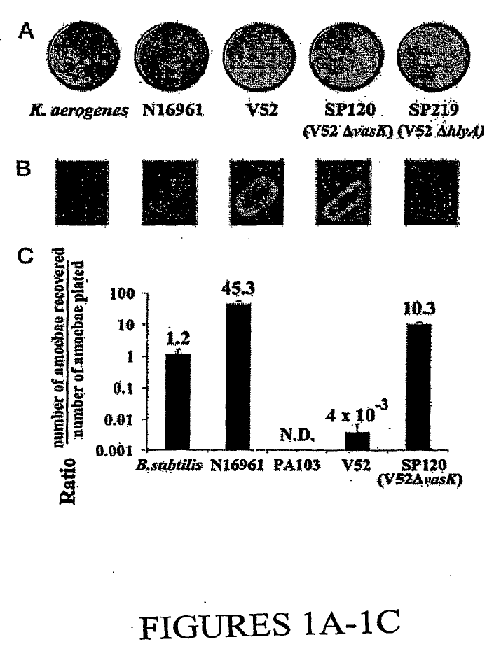

Bacterial predation by Dictyostelium is easily scored by plating individual amoebae on nutrient agar plates seeded with bacterial cells. Successful predation by the amoebae is visualized by the appearance of clear plaques corresponding to zones where actively feeding and replicating amoebae have phagocytosed and killed bacteria. An absence of plaques indicates that the bacterial species being tested displays a “virulent” phenotype on Dictyostelium, by either evading amoeboid killing or actively killing Dictyostelium. As shown in FIG. 1A, Dictyostelium amoebae readily plaque on Klebsiella aerogenes and an O1 serogroup strain of V. cholerae (N16961). In contrast, amoebae plated on the O37 serogroup V. cholerae strain V52 are killed and fail to form plaques, indicating that this non-O1, non-O139 strain expresses virulence factors active on Dictyostelium (see below). The virulence of this isolate for humans is evidenced by the fact that it was isolated from a victim of a large outbreak of diarrheal disease occurring in 1968 that caused 125 deaths in Sudan.

### Identification of VAS Genes

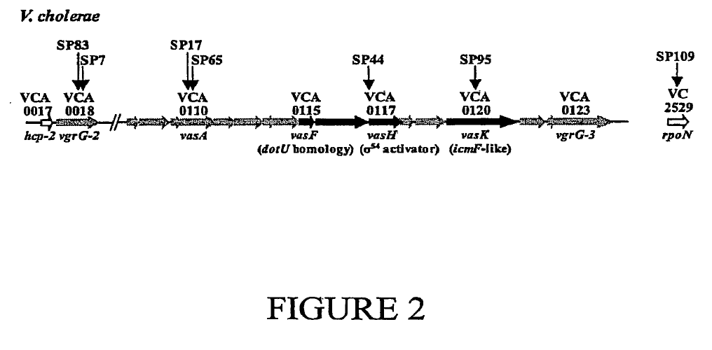

Transposon mutagenesis was used to define the genes encoding Dictyostelium virulence in V52. In brief, a library of V52 cells carrying random insertions of TnAraOut was screened for colonies with a predation-sensitive phenotype on plates containing a large excess of amoebae. Such bacterial mutants formed notched colonies that reflect the active destruction of bacterial cells by feeding amoebae. Virulent wild-type V52 forms smooth, uniformly round colonies because of their resistance to Dictyostelium predation. Dictyostelium-attenuated mutants were purified from notched colonies and characterized for their cytotoxicity toward amoebae in a quantitative assay. Amoebae were mixed with wild-type or mutant bacteria and plated on nutrient agar plates. Bacterial lawns were harvested after 24 h, and surviving amoebae were enumerated by plaque assays in lawns of Escherichia coli strain B/r. As shown in FIG. 1C, wild-type strain V52 causes a 250-fold reduction in the recovery of viable amoebae. A nonpathogenic Bacillus subtilis strain, which does not support Dictyostelium replication, caused no detectable decrease in amoebae viability, whereas P. aeruginosa actively killed amoebae as reported. The low numbers of amoebae recovered from lawns of V52 is therefore caused by active killing by V52 and not starvation. In contrast, the isogenic deletion mutant SP120, which is impaired in the VasK gene, one of the genes that emerged from our mutagenesis screen (see below), lost its virulence, and Dictyostelium efficiently used this mutant as a bacterial substrate at efficiencies comparable to K. aerogenes (FIG. 1A). The most interesting group of genes we identified as being involved in Dictyostelium virulence includes vasA (VCA0110), vasH (VCA0117), and vasK (VCA0120), which are all closely linked on the V. cholerae small chromosome (FIG. 2 and Table 1). Two Dictyostelium-attenuated mutants, SP17 and SP65, carried independent TnAraOut insertions in gene vasA, which is a homolog of impG of Rhizobium leguminosarum, a gene involved in plant root infection by this bacterial species. Another mutant, SP95, carries an insertion in the VasK gene, which encodes a homolog of icmF, a gene involved in intracellular replication of L. pneumophila (see below). vasA and vasK flank vasH, which encodes a predicted activator of Sigma-54, an alternative subunit of RNA polymerase. vasH is disrupted in attenuated mutant SP44 (FIG. 2), and we confirmed that a deletion of vasH in V52 mutant strain SP117 also produced a Dictyostelium-attenuated phenotype. Interestingly, the Dictyostelium-attenuated mutant SP109 carries a TnAraOut insertion on the large chromosome in the gene that encodes Sigma-54 (VC2529). These results suggest that the vasH product and Sigma-54 collaborate to control transcriptional expression of one or more of the Dictyostelium virulence genes expressed by V52.

TABLE 1

| | | | Distance | | BLASTP* | |
|---|---|---|---|---|---|---|
| | | VC | from ATG | | (bits/E | Function ascribed to |
| Strain | Gene | number | (gene length) | Homology | value) | homologue |
| SP17 | vasA | VCA0110 | +510 (1,770) | RL impG | 242/3e−62 | Impaired Rhizobium |
| | | | | | | plant infection |
| SP65 | vasA | VCA0110 | +704 (1,770) | RL impG | 242/3e−62 | See above |
| SP44 | vasH | VCA0117 | +96 (1,593) | EC rtcR | 133/1e−31 | Sigma-54 dependent |
| | | | | | | activator in E. coli |
| SP95 | vasK | VCA0120 | +1477 (3,546) | LP icmF | 154/6e−38 | Type IV protein |
| | | | | | | secretion in L. pneumophila |
| SP7 | vgrG2 | VCA0018 | +985 (2,085) | EC vgrG | 417/8e−117 | Homologous to VgrG-1 |
| | | | | | | that catalyzes actin- |
| | | | | | | crosslinking in |
| | | | | | | eukaryotic cells |
| SP83 | vgrG2 | VCA0018 | +985 (2,085) | EC vgrG | 417/8e−117 | See above |
| SP109 | rpoN | VC2529 | +338 (1,464) | VC rpoN | 862/0.0 | Alternative Sigma-54 |
| | | | | | | subunit of RNA |
| | | | | | | polymerase |

RL, R. leguminosarum;
EC, E. coli;
LP, L. pneumophila;
VC, V. cholerae.
*Position-specific iterated and pattern-hit initiated BLAST (PSI- and PHI-BLAST) statistics. Bits, normalized raw alignment score; E value, expectation value.

Whole-genome microarray-based transcriptome analysis showed that transcriptional expression of two genes, hcp-1 (VC1415) and hcp-2 (VCA0017), was reduced 10-fold in the vasH deletion mutant SP117 compared with wild-type V52. This observation is consistent with the fact that these two genes are required for Dictyostelium cytotoxicity (see below). hcp-1 and hcp-2 both encode an identical protein corresponding to hemolysin-coregulated protein (Hcp), a secreted V. cholerae protein that is coexpressed with HlyA hemolysin. However, Dictyostelium-attenuated mutants were found to still express and secrete HlyA (FIG. 1B) and the hlyA mutant SP219 was fully virulent on Dictyostelium (FIG. 1A). Two genes in the cluster, vasK (VCA0120) and vasF (VCA0115), show a high degree of similarity to icmF and icmH (dotU), two genes found in L. pneumophila. In L. pneumophila, these genes are nonessential components of the type IV secretion system (T4SS) required for cytotoxicity of L. pneumophila toward mammalian and D. discoideum cells. IcmF and IcmH (DotU) are thought to be accessory proteins that work in concert to improve the efficiency of T4SS translocation of bacterial effector proteins into the cytosol of eukaryotic target cells. VasK and VasF may also cooperate in V. cholerae, because vasF and vasK deletion mutants also show a Dictyostelium-attenuated phenotype. No other V52 mutant carried insertions in a gene with homology to the group of genes including dotA, dotB, dotC, dotD, icmB, icmC, icmD, icmE, icmG, icmJ, icmK, icmL, icmM, icmN, icmO, icmP, icmQ, icmR, icmS, icmT, icmV, icmW, and icmX most of which have been shown to be absolutely required for function of the Legionella T4SS. To determine whether V52 has a T4SS gene cluster, we sequenced the genome of this strain to times 6.5 coverage. Careful annotation of the sequence found no evidence for the existence of genes encoding homologs of dotA, dotB, dotC, dotD, icmB, icmC, icmD, icmE, icmG, icmJ, icmK, icmL, icmM, icmN, icmO, icmP, icmQ, icmR, icmS, icmT, icmV, icmW, and icmX and thus we conclude that V52 does not carry a recognizable T4SS gene cluster. Also, unlike other non-O1, non-O139 V. cholerae strains, the V52 genome does not encode a T3SS other than the typical one required for flagella biosynthesis. Thus, it is also notable that no Dictyostelium-attenuated mutants were found in any gene known to be involved in flagellar biosynthesis.

### The VAS Pathway is Responsible for the Secretion of Proteins Lacking N-Terminal Leader Sequences

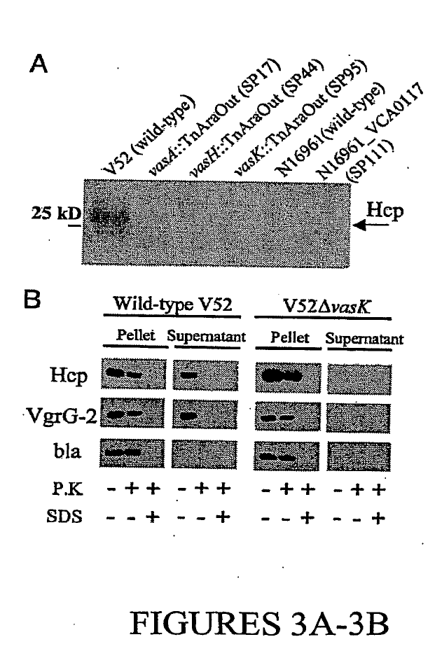

Because some T3SS and T4SS pathways transport their effector proteins into the bacterial culture supernatant fluids in the absence of eukaryotic target cells, we analyzed culture supernatant fluids of V52 for evidence of such protein export. SDS-PAGE was used to visualize the proteins secreted by V52 and three different Dictyostelium-attenuated mutants. We also analyzed supernatant fluids from Dictyostelium-sensitive strain N16961 and the isogenic vasH-deletion mutant SP111. As shown in FIG. 3A, a 28-kDa band that was identified by MS as Hcp appeared as an abundant protein in the supernatant fluid of V52. Interestingly, Hcp was absent in supernatants of V52 mutants with transposon insertions in vasA, vasH, and vasK (strains SP17, SP44, and SP95, respectively), as well as wild-type N16961 and its vasH-deletion mutant SP111. Accordingly, we constructed hcp-1 and hcp-2 single- and double-deletion mutants and found that only a double-deletion mutant was avirulent toward Dictyostelium. Virulence was restored when a plasmid allowing isopropyl β-D-thiogalactoside-inducible expression of Hcp was introduced. Thus, both hcp alleles are functional and Hcp is apparently essential for VAS-mediated amoebae cytotoxicity. It is also of interest that Hcp has been reported to lack a hydrophobic leader peptide and was previously detected with an unprocessed amino terminus in the supernatant fluids of V. cholerae. Analysis of culture supernatant fluids of V52 by electron-spray ionization liquid chromatography tandem MS identified additional proteins in supernatants of V52 and Dictyostelium-attenuated mutants. Strains carrying mutations in vasA, vasH, and vasK were still able to secrete proteins with hydrophobic amino-terminal signal sequences, namely chitinase, neuraminidase, PrtV protease, and HlyA hemolysin. These proteins are known to be secreted by type I and type II secretion pathways. Critically, V52 secreted four proteins that could not be detected in supernatants of N16961, namely Hcp, VgrG-1, VgrG-2, and VgrG-3. These four proteins lack identifiable hydrophobic amino-terminal signal sequences. In contrast, Dictyostelium-attenuated mutants SP17, SP44, SP83, SP95, and SP109 also showed undetectable levels of Hcp, VgrG-1, VgrG-2, and VgrG-3 in their culture supernatant fluids. In fact, in our initial mutant screen, we isolated two independent Dictyostelium-attenuated mutants, SP7 and SP83, that each carry a transposon insertion in the VgrG-2 gene. These results strongly suggest that VgrG-2 is also an essential component in the pathway leading to cytotoxicity of Dictyostelium amoebae. In conclusion, the V. cholerae VAS pathway does not appear to be required for secretion of any protein with hydrophobic amino terminus signal sequences, but is essential for secretion of Hcp, VgrG-1, VgrG-2, and VgrG-3, all of which lack such signal sequences.

We did not detect the predicted protein products of vasA and vasK in the culture supernatant of V52. These data suggest that these protein products are not secreted but are nonetheless required for the function of a secretion pathway that transports Hcp, VgrG-1, VgrG-2, and VgrG-3 to the exterior of bacterial cells. If this model is correct, epitope-tagged versions of Hcp-2 and VgrG-2 should be secreted by wild-type V52 but not by an isogenic vasK mutant. To follow VasK-mediated secretion, we introduced a plasmid that allows arabinose-induced expression of Hcp-2 and VgrG-2 tagged with a vesicular stomatitis virus glycoprotein epitope at their C termini into wild-type V52 and the isogenic vasK deletion mutant SP120. Only wild-type V52 was able to secrete these two tagged proteins into the culture supernatant (FIG. 3B). Both V52 and the vasK mutant SP120 produced equal amounts of Hcp-2 and VgrG-2 that accumulated inside the bacteria cells as evidenced by their resistance to proteolytic degradation when cells were treated with proteinase K (FIG. 3B). Thus, proteins Hcp-2 and VgrG-2 rely on VasK for their extracellular secretion.

### VAS Genes are Highly Regulated

Although all V. cholerae strains analyzed so far by microarray analysis carry DNA corresponding to VAS, hcp, and vgrG genes, our current data suggest that, unlike V52, most O1 and O139 strains, like N16961, are permissive for Dictyostelium predation under the in vitro conditions examined so far. This discrepancy may be explained by the results of our microarray analysis that show that the hcp genes are highly expressed in V52 compared with N16961 under in vitro conditions that stimulate VAS-dependent Hcp secretion by V52. In addition, N16961 is unable to secrete Hcp into culture fluids even when this gene is expressed via a heterologous promoter. These experiments suggest that some V. cholerae strains with VAS gene clusters are unable to use the VAS secretion pathway perhaps because their Vas, Hcp, or VgrG genes are not properly regulated. This hypothesis is supported by the fact that VAS genes are tightly regulated in vivo. For example, vasK has been reported to be an in vivo-induced gene in a rabbit model for cholera, whereas its homolog in Salmonella enterica, sciS, is in vivo-induced in macrophages. Other VAS-related gene products have been identified as antigens recognized by catfish infected with Edwardsiella ictaluri, suggesting they are also in vivo-induced. Thus, transcriptional regulation in vivo may be a common characteristic of VAS genes and their homologues.

### Hcp Mediates Translocation of VgrG-1 and VgrG-2

We have identified two other non-O1, non-O139 V. cholerae strains, SCE223 and SCE226, that are virulent for Dictyostelium and express and secrete Hcp under in vitro conditions. As in V52, in-frame vasK deletions in these two strains rendered them sensitive to predation by Dictyostelium and blocked Hcp export. Thus, expression and secretion of Hcp by a VAS pathway correlates with Dictyostelium virulence for other strains of V. cholerae besides V52. Because Hcp appears to be a central component of the VAS pathway, we asked whether Hcp was essential for the secretion of VgrG-1 and VgrG-2. When a plasmid that allows arabinose-induced expression of Hcp was introduced into a V52 mutant strain with deletions in both Hcp genes, VgrG-1 and VgrG-2 could be detected in culture supernatants by MS only when Hcp expression was induced. Thus, Hcp is both secreted by the VAS pathway and required for the extracellular secretion of other proteins like VgrG-1 and VgrG-2.

V. cholerae Uses the VAS Pathway to Mediate Virulence Toward J774 Macrophages

By analogy to T3SS and T4SS, the extracellular secretion of Hcp, VgrG-1, and VgrG-2 may actually reflect a more complex process that involves the translocation of these proteins by V. cholerae into eukaryotic target cells. Recently, Sheahan et al. (Proc. Natl. Acad. Sci. USA 101:9798-9803, 2004) reported that VgrG-1 and the V. cholerae RtxA toxin share a subdomain that mediates actin covalent crosslinking and cytotoxicity when expressed in the cytosol of mammalian cells. The extracellular cytotoxin RtxA, however, is not required for Dictyostelium virulence, because a rtxA mutant of V52 still kills amoebae. Thus, the actin-crosslinking activity of VgrG-1 suggests that this protein might be a cytotoxic effector transported into mammalian target cells by the VAS secretion pathway.

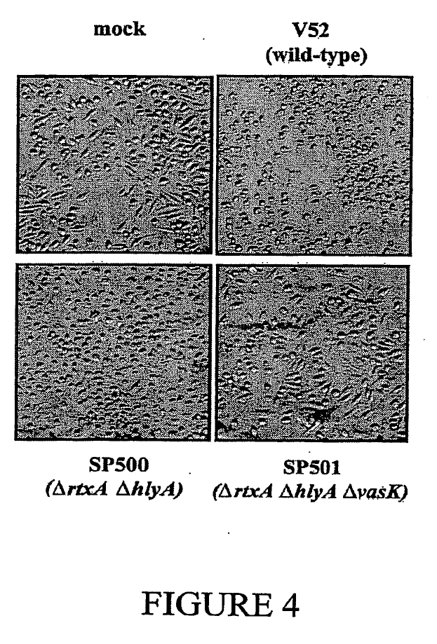

We asked whether VAS-mediated secretion was associated with V. cholerae-mediated cytotoxicity toward a mammalian macrophage cell line. Because several different V. cholerae toxins, including RtxA and HlyA, can disrupt mammalian cell structures, we examined the effect of a vasK mutation in the context of mutations in these other two factors. As shown in FIG. 4, the morphology of J774 macrophages is disrupted within 2 h of exposure to live V52. Mutant SP501 disrupted in rxtA, hlyA, and vasK lost all detectable cytotoxicity toward J774 cells, whereas its parent strain SP500, disrupted in only rtxA and hlyA, retained this property. Interestingly, media supernatants from wells infected with SP500 showed no cell rounding activity when added to wells containing uninfected J774 cells, suggesting bacterial-macrophage cell-cell contact is a requisite for SP500 cytotoxicity. In conclusion, vasK and the VAS-dependent secretion pathway contribute significantly to the cytotoxicity that V. cholerae displays toward this mammalian macrophage cell line in a cell-contact-dependent manner.

The genes of the IAHP gene cluster, together with other Vas, Hcp, and VgrG genes, likely encode the T6SS apparatus and several of its translocated effectors. Because so many pathogenic Gram-negative bacterial species carry VAS gene clusters, we predict that the primary function of the T6SS is to mediate extracellular export of virulence factors and their translocation into target eukaryotic cells. Because this transport will likely have deleterious effects on the host, the components of the T6SS constitute exciting candidates for the development of preventative or therapeutic vaccines and targets for antimicrobial drug development.

### Materials and Methods

The experiments described in Example 1 were performed using the following materials and methods.

Strains and Culture Conditions

D. discoideum strain AX3 was used in all experiments. AX3 was grown in liquid HL/5 cultures or in lawns of K. aerogenes on SM/5 plates, as described by Sussman (Methods Cell. Biol. 28:9-29, 1987). V. cholerae O37 serogroup strain V52 and El Tor biotype strain N16961 were used in all experiments. E. coli strains DH5α-λpir and SM10λpir were used for cloning and mating, respectively. All bacterial strains were grown in Luria broth (LB). J774 cells were obtained from the American Type Culture Collection.

Transposon Library of V. cholerae Strain V52

Mariner transposon TnAraOut was introduced into V. cholerae by using DTH2129-2, a derivative of suicide plasmid pNJ17. E. coli BW20767 was used to mobilize DTH2129-2 by conjugation into streptomycin-resistant V. cholerae strain V52 by incubating donor and recipient at a 10:1 ratio on LB agar for 60 min at 37° C. Bacteria were collected, and dilutions were plated on LB agar containing 100 μg/ml kanamycin and 100 μg/ml streptomycin to select for V. cholerae clones carrying TnAraOut.

Isolation of Dictyostelium-Attenuated V. cholerae

Amoebae (5×10⁶) were mixed with 1×10³ TnAraOut mutants of V. cholerae strain V52 and plated onto SM/5 plates containing 100 μg/ml kanamycin. Plates were incubated at 22° C. for 3 days and then scored for notched V. cholerae colonies formed by Dictyostelium-attenuated V. cholerae mutants. Bacteria were restreaked on SM/5 plates containing 5 μg/ml blasticidin to kill amoebae.

Plaque Assay

Bacteria were grown in LB for 16 h, pelleted by centrifugation, washed once, and resuspended in SorC (16.7 mM Na₂H/KH₂PO₄/50 μM CaCl₂, pH 6.0) at a final OD of 5.5 at 600 nm. D. discoideum cells from midlogarithmic cultures were collected by centrifugation, washed once with SorC, and added to the bacterial suspensions at a final concentration of 5×10² cells per ml suspension; 0.2 ml of this mixture was plated on SM/5 plates and allowed to dry under a sterile flow of air. Plates were incubated for 3-5 days and examined for plaques formed by Dictyostelium amoebae.

Plate Killing Assay

Bacterial strains were plated with D. discoideum on SM/5 plates as described for the plaque assay. After 24 h, bacterial lawns containing amoebae were collected and enumerated by plating with tetracycline-resistant E. coli B/r on SM/5 plates containing 30 μg/ml tetracycline. Plaques were counted 3 days later.

Secretion Assay

Hcp was isolated from midlog cultures of V. cholerae. Briefly, culture supernatants were sterilized by passing through a 0.2-μm filter (Millipore), and proteins were precipitated with trichloroacidic acid (TCA) and subjected to 4-12% gradient SDS-PAGE. Extracellular secretion of epitope-tagged substrates was determined by growing V. cholerae strains maintaining a plasmid with tagged Hcp-2 and VgrG-2 fused to the arabinose-inducible PBAD promoter in LB containing 0.1% arabinose. Midlog cultures were harvested and cells were isolated by centrifugation. Cells and 0.2-μm filtered supernatants were left untreated or incubated with either 0.1 mg/ml proteinase K in the presence or absence of 1% SDS. After a 20-min incubation at room temperature, protease inhibitor PMSF (final concentration of 1 mM) was added to all samples, and proteins were precipitated with TCA, solubilized in sample buffer, and separated on a SDS-PAGE for immunoblotting with vesicular stomatitis virus glycoprotein antisera. The quality of pellet and supernatant fractionation was determined by localizing periplasmic β-lactamase.

Cell Rounding of J774 Macrophages

Bacterial midlog cultures grown in LB were washed with PBS and added to adherent J774 cells (multiplicity of infection ˜10) cultured in advanced DMEM (GIBCO) containing 10% FCS. Cells were infected for 2 h at which time supernatants were replaced with 3% formaldehyde to fix adherent cells. Saved supernatants were sterilized by centrifugation and treatment with 0.1 mg/ml gentamicin for 30 min at 37° C. and transferred to wells containing uninfected J774 cells. Cell rounding was monitored with a Nikon Diaphot 200 inverted microscope equipped with computer interface.

### EXAMPLE 2

We now show that P. aeruginosa IAHP-I is required for secretion of Hcp1. Furthermore, we show that IAHP-I-dependent secretion of Hcp1 is strongly repressed by RetS, a hybrid two-component regulator of several known virulence pathways. Using a fluorescent tag appended to a conserved IAHP gene product with strong homology to the AAA+ family ATPase ClpB, we provide data suggesting that the IAHP-I locus encodes a secretory apparatus localized to discrete foci within the cell. In addition, we determined the 1.95 Å crystal structure of Hcp1 and found it to be a hexamer with a large (42 Å) internal diameter. Our analysis of the crystal structure suggests that the protein forms interactions on both its faces and likely facilitates the passive diffusion of a macromolecular species.

Our findings are discussed in more detail below.

P. aeruginosa Possesses Three IAHP Loci

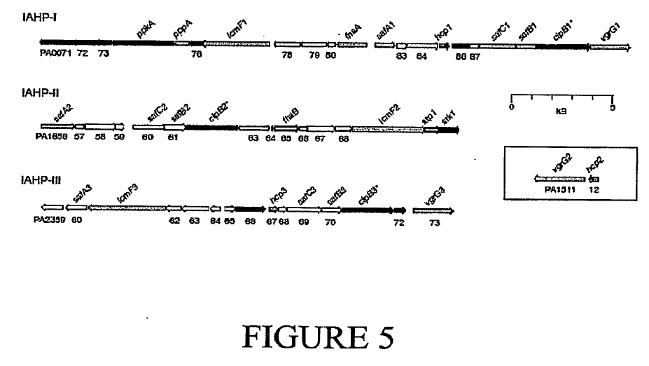

P. aeruginosa possesses three unlinked IAHP loci (FIG. 5). We identified several features shared between P. aeruginosa IAHP loci and known bacterial secretion systems. Each of these loci contains a putative open reading frame (ORF) encoding a protein with a high degree of homology to IcmF (FIG. 5, icmF1-3). IcmF is a predicted membrane protein that was originally linked to protein secretion by virtue of its role in the L. pneumophila dot/icm type IVB secretion system. Moreover, we show herein that the IcmF homolog present in the V. cholerae IAHP locus has been shown to be required for the secretion of Hcp and VgrG1-3 (see above). A predicted ORF encoding a protein with strong homology to the AAA+ family protein ClpB is also present in each of the IAHP loci (FIG. 5, clpB1-3*). AAA+ family ATPases are required for the function of several bacterial secretion systems, where their ability to couple the energy derived from ATP hydrolysis to movement is thought to provide the force for substrate translocation. A cluster of genes that display weak homology to a Salmonella enterica type IV fimbrial assembly cluster (Salmonella atypical fimbria-saf) was previously detected in the P. aeruginosa IAHP loci. Homologs of sajB and safC, which are predicted to serve as the chaperone and outer membrane usher in the Salmonella fimbrial assembly cluster, respectively, are located upstream of the clpB*gene in each locus (FIG. 5). A homolog of safA, the predicted structural subunit in the Salmonella cluster, is also detectable in each locus. While the homology to fimbrial assembly genes is weak, the presence of three such homologs in each of the loci may be indicative of a common ancestral origin for these gene clusters. IAHP-I and III contain predicted ORFs encoding proteins with homology to Hcp (FIG. 5, hcp1 and hcp3) and the RHS element-associated VgrG (FIG. 5, vgrG1 and vgrG3). Third homologs of both of these proteins are adjacent to each other and are encoded for elsewhere in the genome (FIG. 5, boxed). Genes encoding vgrG homologs occur in regions with a high propensity for genetic rearrangements in both E. coli and P. aeruginosa, although the significance of this correlation is unclear. In V. cholerae, the expression of hcp is regulated coordinately with hemolysin (hlyA) by the HlyU transcription factor. We show above that that V. cholerae Hcp and VgrG are secreted in an IAHP-dependent manner, and furthermore, that both of these proteins are required for full virulence of the organism against D. discoideum.

### IAHP-I is Required for Hcp1 Secretion and is Regulated by RetS

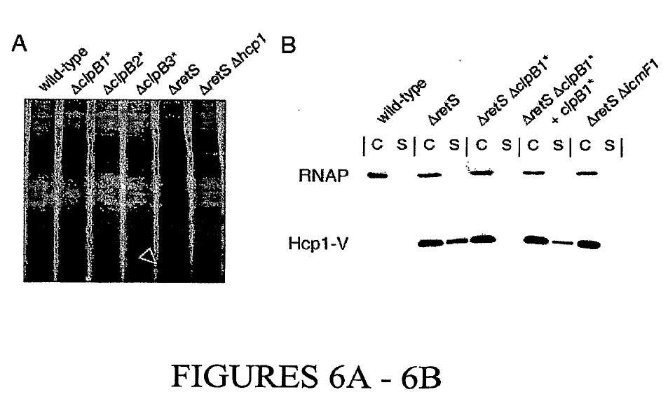

We hypothesized that one or more of the P. aeruginosa IAHP loci might be involved in protein secretion. We tested this hypothesis by generating strains containing in-frame deletions of the clpB homologs (clpB1-3*) within each of the IAHP loci. Our rationale to use clpB* deletions to inactivate each locus was based on its strict conservation in IAHP loci, and on our prediction that the two AAA+ domains of this protein would be a necessary secretory energy source (see below). To identify potential defects in protein secretion, we prepared concentrated secreted protein samples from ΔclpB1-3* and subjected them to SDS-PAGE analysis. Under a variety of growth conditions, this method failed to identify reproducible differences in secreted proteins (FIG. 6A). Since IAHP loci are found in many pathogens that maintain close interactions with eukaryotic hosts, we reasoned that in vitro growth conditions might repress their function. In support of this notion, a prior microarray study showed that the P. aeruginosa IAHP-I locus is highly repressed by the hybrid two-component regulator RetS (Regulator of Exopolysaccharide and Type III Secretion). Interestingly, RetS appears to globally regulate the phenotypic morphogenesis of P. aeruginosa from acute to chronic phase infection.

To test the prediction that mutation of RetS would activate IAHP-I and thereby lead to protein secretion, we deleted retS from PA01 (ΔretS). Consistent with the global regulatory activity of RetS, several differences in the secreted protein profiles of wild-type and ΔretS were apparent by SDS-PAGE (FIG. 6A). The most apparent difference, however, was the abundant secretion of a small protein by ΔretS that was not detected in wild-type (FIG. 6A). In-gel digestion followed by tandem mass spectrometry identified the protein as Hcp1 (PA0085). This was further confirmed by generating an in-frame deletion of hcp1 in ΔretS (ΔretS Δhcp1). SDS-PAGE analysis of supernatants prepared from this strain indicated that the strain was devoid of the overproduced protein (FIG. 6A). Notably, Hcp1 resides in one of the two operons predicted to encode IAHP-I, further suggesting a functional link between IAHP-I and Hcp1 secretion (FIG. 5).

In order to more quantitatively assess the production and localization of Hcp1, we constructed a C-terminal chromosomal fusion of hcp1 to the VSV-G epitope (hcp1-V). Western blot analyses of cellular and secreted protein fractions from hcp1-V and ΔretS hcp1-V were consistent with results obtained from cells expressing native Hcp1:Hcp 1-V was detected in culture supernatants devoid of cell-associated contaminants, and the secretion of Hcp1-V was highly repressed by RetS (FIG. 6B). Also, with this more sensitive assay, we detected low levels of Hcp1-V secretion in wild-type supernatants. To test our prediction that clpB1* would be required for secretion, we constructed an in-frame deletion of clpB1* in ΔretS hcp1-V and assayed for Hcp1-V secretion by western blot (FIG. 6B). This analysis indicated that Hcp1-V is produced intracellularly in ΔclpB1* ΔretS, whereas the secretion of Hcp1-V is abrogated. Genetic complementation of ΔclpB1* ΔretS confirmed that the Hcp 1-V secretion defect was not due to a polar effect (FIG. 6B). In several instances, disruption of the IAHP icmF homolog has been sufficient to reveal a phenotype. Indeed, two independent transposon insertions in the icmF homolog of V. cholerae were identified in a screen for mutants attenuated against D. discoideum. Analysis of culture supernatants from these icmF mutants showed an accompanying defect in Hcp secretion. To determine whether P. aeruginosa IAHP-I icmF1 was similarly required for Hcp1 secretion, we constructed an in-frame deletion of this gene in ΔretS hcp1-V and probed for the presence of Hcp1 in cell and supernatant fractions (FIG. 6B). While Hcp1 was readily detected in the cellular fraction, secreted Hcp1 could not be detected. These data indicate that icmF1 is required for Hcp1 secretion.

### ClpB1-3* are not Required for Thermotolerance

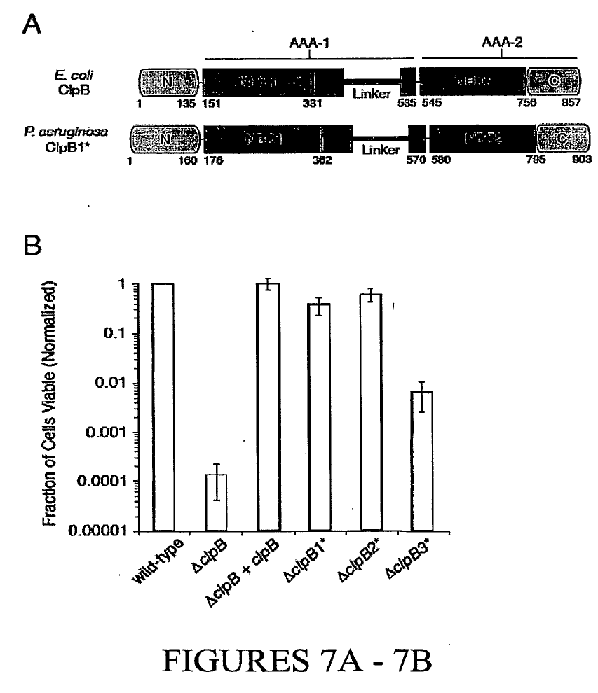

As discussed above, each IAHP loci contains a gene predicted to encode a protein with a high degree of sequence homology to ClpB (FIG. 7A, see legend). ClpB is a AAA+ family ATPase that maintains cellular viability during stressful conditions by functioning as a protein disaggregase. The high-resolution crystal structure of the ClpB monomer and an electron microscopic (EM) reconstruction have previously shown that the protein oligomerizes to form a hexameric ring. Based on these structural data and compelling biochemical observations, a molecular mechanism of ClpB-dependent protein disaggregation has been proposed in which ClpB translocates proteins unidirectionally in an energy dependent manner, and protein transport occurs through its central channel. These properties of ClpB, combined with its high degree of sequence homology to ClpB*, prompted us to speculate that ClpB* may function in an analogous manner. However, rather than functioning as a disaggregase, we hypothesize that ClpB* serves as a structural component and energy dependent protein translocase for putative IAHP secretory apparatuses.

Several studies in various bacteria have shown that clpB mutants display an exquisite sensitivity to elevated temperatures. We used this phenotype as a measure to determine whether the ClpB* proteins are involved in cellular processes similar to those of ClpB. The role of ClpB in the thermotolerance of P. aeruginosa had not previously been investigated; therefore, we generated an in-frame deletion of the gene (ΔclpB) and tested the ability of this strain to survive exposure to thermal stress (FIG. 7B). Consistent with studies in other organisms, ΔclpB was approximately 10,000 fold more susceptible than wild-type to a 25 minute heat pulse at 55° C. We next tested each of the clpB* mutants under the same conditions. In this assay, ΔclpB1* and ΔclpB2* displayed essentially wild-type levels of resistance to thermal challenge. A reproducible defect was observed for ΔclpB3*, however this defect is 100-fold less than that of ΔclpB (FIG. 7B). Furthermore, we noted that ΔclpB3* was also highly sensitive to other stresses, for example osmotic stress. Therefore, we propose that the role of ClpB3* is distinct from that of ClpB. Based on these results, we conclude that in contrast to ClpB, the ClpB* proteins are not required for P. aeruginosa thermotolerance.

### ClpB1* Localizes to Punctate Foci in an IcmF1- and Hcp1-Dependent Manner

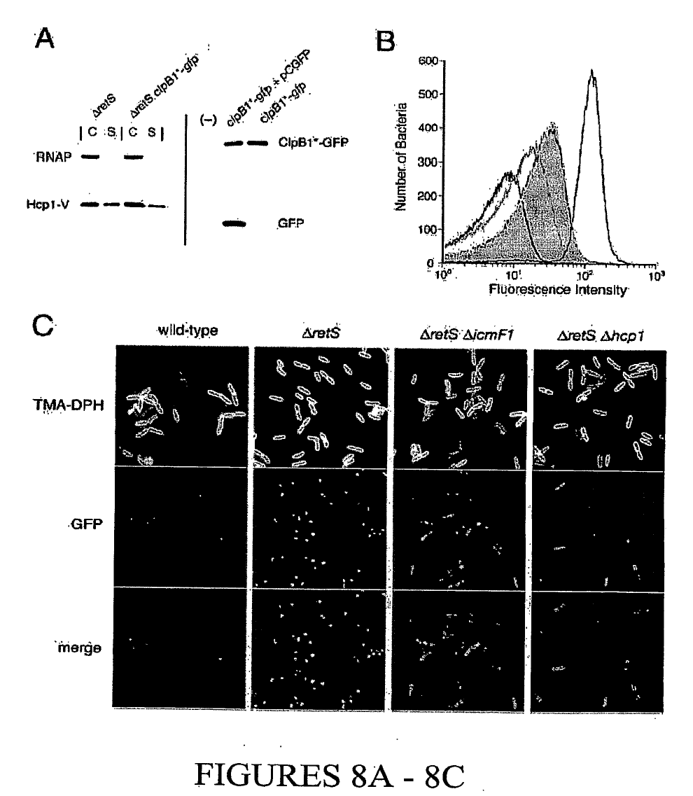

If ClpB1* forms an essential component of the IAHP-I secretion machinery, we hypothesized that this role would be reflected in its subcellular localization. To assess the subcellular localization of ClpB1*, we generated a strain carrying a chromosomal C-terminal fusion of the green fluorescent protein (GFP) to clpB1* (clpB1*-gfp) in the ΔretS background. Since GFP fusions often interfere with function, we first tested whether fusion of GFP to ClpB1* affected Hcp1 secretion. For this analysis, we generated a strain harboring both clpB1*gfp and hcp1-V chromosomal fusions in the ΔretS background. Western blot analysis of culture supernatants from this strain indicated that Hcp1-V secretion was not affected by the GFP fusion (FIG. 8A, left). One possible explanation for this finding is that proteolytic activity liberates GFP from ClpB1*, and it is this form lacking GFP which is functional. To explore this possibility, we probed cellular protein samples for the release of GFP from ClpB1*. Western blot analysis with a GFP-specific antibody failed to detect degradation of the ClpB1*-GFP fusion, suggesting that the intact fusion is fully active (FIG. 8B, right). To assay the localization of ClpB1*, we visualized the ΔretS clpB1*-gfp strain by fluorescence microscopy (FM). Interestingly, the fluorescent signal in a large fraction of these cells was restricted to single punctate foci (FIG. 8C). When GFP was expressed alone, the fluorescent signal was uniformly distributed in the cell. To assess whether this pattern of localization was an artifact of the ΔretS background, we examined the localization of ClpB1*-GFP in wild-type. While less intense, a similar pattern of punctate localization was observed in the wild-type background (FIG. 8C). Localization to discrete foci can be indicative of the association of a protein with a large macromolecular complex. For example, this pattern of localization has been observed for the AAA+ family ATPases of several secretion apparatuses. To establish whether the punctate localization of ClpB1* held a similar physiological significance, we deleted icmF1 and measured the resulting effect on ClpB1* localization. The number of cells with focal localization of ClpB1*-GFP was dramatically decreased in ΔretS ΔicmF1 clpB1*-gfp. Rather, the fluorescent signal from these cells was most often evenly distributed across the cell, or in some cases weak foci were observed (FIG. 8C). This difference in localization was not due to proteolytic degradation of ClpB1*-GFP. We also performed flow cytometry on ΔretS ΔicmF1 clpB1*-gfp to probe whether the appearance of these cells could be explained by a lower level of ClpB1*-GFP expression. This analysis indicated that ΔretS clpB1*-gfp and ΔretS ΔicmF1 clpB1*-gfp express equal levels of ClpB1*-GFP. These results demonstrate that IcmF1 is required for ClpB1* localization. In the case of type IV pili, assembly of the pilin secretion complex is dependent on the presence of the major secreted protein, PilA. Given that some genetic constituents of the type IV pili assembly pathway appear to be conserved in the IAHP-I locus, we questioned whether Hcp1 may similarly be required for the assembly of a putative IAHPI secretion apparatus. This hypothesis was addressed by generating an Hcp1 deletion in the ΔretS clpB1*-GFP strain and assaying for ClpB1*-GFP localization by FM. In general, these cells displayed a diffuse localization of ClpB1*-GFP similar to that of ΔretS ΔicmF1 clpB1*-gfp, although the frequency of residual punctate foci was decreased (FIG. 8C).

Taken together, these data support the notion that ClpB1* localization to punctuate foci is functionally linked to Hcp secretion and begin to provide evidence for an IAHP-I-encoded secretory apparatus. It is interesting that hcp1 deletion results in a more complete disruption of ClpB1* localization than a corresponding icmF1 deletion. We hypothesize that IcmF is required for efficient assembly of the apparatus, which is consistent with its role in the L. pneumophila type IVB secretion system, whereas Hcp1, as the major IAHP-I secreted protein, is absolutely required for IAHP-I assembly.

Hcp1 Forms a Hexameric Ring with a Large Internal Diameter

Hcp shares little detectable sequence homology with proteins of known structure; therefore, in an effort to gain insight into the function of Hcp, we determined the X-ray crystal structure of P. aeruginosa Hcp1 to a resolution of 1.95 Å. Hcp1 crystallized in the P6 spacegroup with three nearly identical monomers in the asymmetric unit.

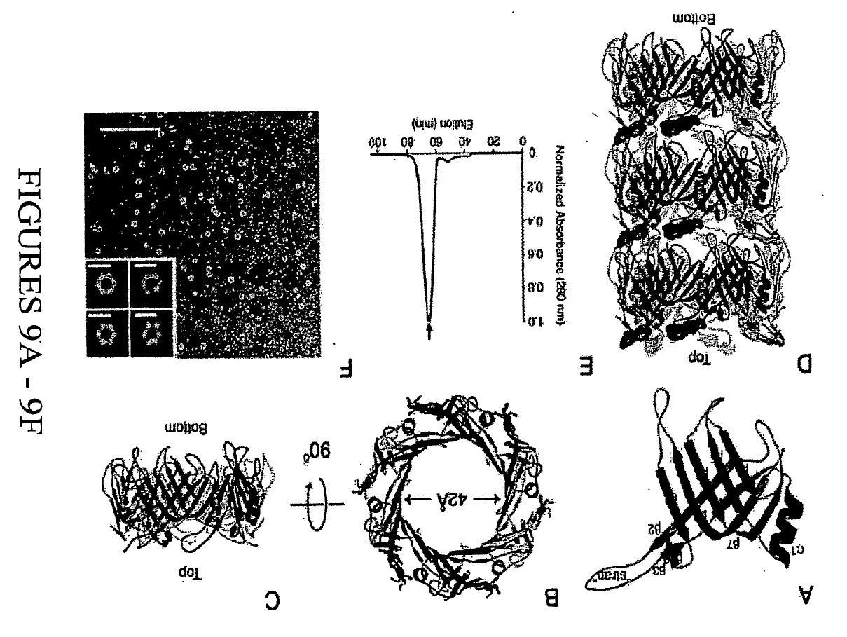

The secondary structure of the Hcp1 monomer consists of 10 β-strands and a single α-helix (FIG. 9A). The β-strands are organized into two anti-parallel β-sheets that pack against each other, forming the core of the molecule. At one end of the β-sandwich, the two sheets separate to form extensive interactions with the single α-helix of the structure. As discussed below, an extended loop, which we term the “strap,” protrudes from the other end of the 1-sandwich. A search of the protein structure database revealed that the closest structural homologs of Hcp1 are oxidoreductase proteins that bind the flavin nucleotide. Though the overall structure of Hcp1 largely conforms to the flavin-binding domain fold, a detailed comparison between several such proteins and Hcp1 demonstrated that the nucleotide-binding pocket of these proteins is not maintained in Hcp1.

Within the P6 crystal lattice we obtained, Hcp1 is organized into hexameric rings that stack end-on-end to form tubes (FIG. 9B-9D). Although the subunit contacts required to assemble the hexameric form are observed between two of the three monomers in the asymmetric unit, we sought to provide biochemical evidence that this hexameric form of Hcp1 is populated under physiological conditions. To determine the oligomeric state of Hcp1 in solution, we subjected the purified protein to analytical gel filtration chromatography. Hcp1 eluted as a single species at a mass consistent with that of the hexamer (FIG. 9E). Next, we visualized the organization of this oligomeric species by transmission EM. Micrographs of negatively stained single particles clearly indicated that the predominant form of Hcp1 is a ring assembly with dimensions closely matching those observed in the crystal lattice (FIG. 9F). Furthermore, averaging of approximately 6,000 particles indicated that the rings contained six clearly discernable subunits with pseudo 6-fold symmetry. From these data, We conclude that the hexameric rings found in the Hcp1 crystal structure are physiologically relevant and represent the predominant form of the protein in solution.

Extensive monomer contacts, predominantly hydrophobic in nature, appear to stabilize the Hcp1 hexamer. Approximately 50% of the solvent accessible surface area of each monomer (830 Å2 at each dimer interface) is buried in the hexamer. Most of these contacts are accounted for by the interactions of α1 of one subunit and β-strands 2, 3, and 10 of the adjacent subunit. A second set of significant subunit contacts are mediated by the extended glycine-rich “strap” that protrudes from one subunit and interacts with several residues on the top face of the adjacent subunit (FIG. 9A-9D). This strap contains six glycine residues interrupted by a single alanine. While the role of these glycine residues is not known, it is noteworthy that they are among the most highly conserved Hcp1 residues, yet they are not the strap residues that contact the neighboring protomer. Rather, the glycine residues constitute the region of the strap directed outward from the ring, suggesting that they may be important for mediating interactions with other proteins. Interestingly, conserved residues in this outward-facing region of the strap overlap significantly with residues that form the ring-to-ring stacking interactions in the crystal lattice (FIG. 9D).

Among the protein structures available in the protein structure databank which form multimeric rings, we are not aware of any examples in which the rings stack in the crystal lattice to form a continuous tube as observed in the crystal structure of Hcp1 (FIG. 9D). Several precedents exist for secreted bacterial proteins forming extended tube structures, including pili, flagella, and the type III protein secretion needle. In these structures, however, the major structural subunit polymerizes in a helical fashion, presumably to promote stability. If the tubes we observe in the Hcp1 crystal lattice are biologically relevant, their non-helical, end-on-end stacking would represent a marked departure from this organization.

Hcp1 does not Reside in a Membrane

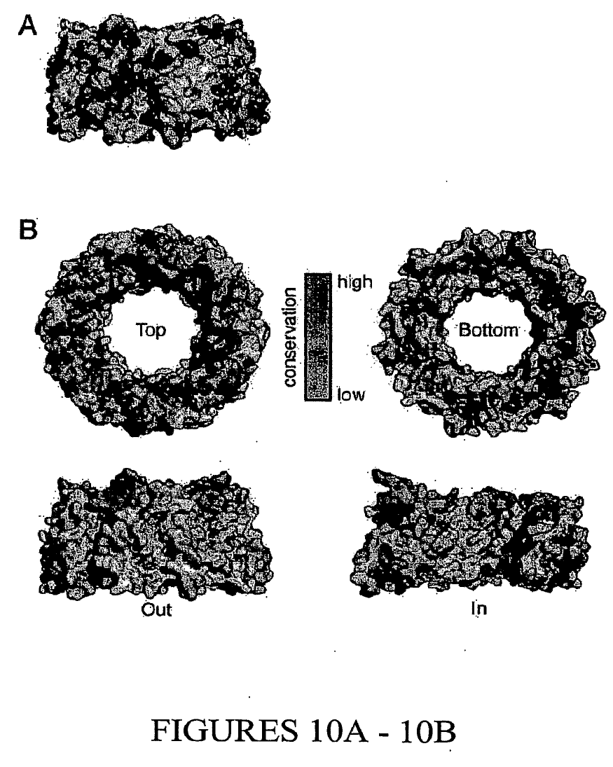

The function of many bacterial secreted proteins is to form pores in host membranes, either to promote lysis of the host cell, or to allow the passage of bacterial effectors into the host cytoplasm. The ring shape of the Hcp1 hexamer, combined with its large internal diameter, prompted us to speculate that the protein may be capable of introducing membrane pores. As a preliminary analysis to assess the feasibility of this notion, we examined the hydropathy of the outer surface of Hcp1. Contrary to known membrane proteins, the outer circumference of Hcp1 does not contain a continuous hydrophobic belt that would accommodate the lipid groups of the membrane (FIG. 10A). Additionally, we employed sensitive voltage-gated artificial bilayer assay to assay directly whether Hcp1 possessed such activity. Under all conditions tested, we were unable to detect pore-forming activity of Hcp1. The combination of these biochemical data and our structural analyses strongly suggests that Hcp does not reside in a membrane.

### Hcp1 Sequence Conservation Suggests Interactions at Both Faces

Structure-based sequence conservation can serve as a powerful predictor of protein interaction interfaces and enzymatic active sites. In an attempt to identify regions of the Hcp1 structure that are important for its biological activity, we generated an alignment of 107 Hcp1 proteins from 43 bacterial species and plotted the degree of conservation of each residue onto the structure. This analysis revealed an interesting pattern of conservation: the most highly conserved surface residues of Hcp1 are found on the top and bottom faces of the protein, while residues located around the inner and outer circumferences are poorly conserved (FIG. 10B). A particularly well-conserved patch of residues occupies the cleft on the bottom face of Hcp1 (residues 15, 16, 26, 60, 63, 88, 89, 169, and 139). Many of these residues mediate critical subunit contacts, perhaps explaining their high degree of conservation. Others such as Lys88, Asp26, and Gln139, are not engaged in hexamer-stabilizing interactions, and hence, it is likely that the conservation of these residues reflects their involvement in the function of Hcp1. The highly conserved residues located on the top face of Hcp1 are almost completely contained to the glycine-rich strap. Not surprisingly given the amino acid composition of this region, the B factors of strap residues are high, and indeed, sufficiently disordered in one protomer of the asymmetric unit that we were unable to interpret the electron density of Gly46. The conservation of such a highly mobile loop at the face of the hexamer suggests that it plays a crucial role in the biological activity of Hcp1. Taken together, the strict conservation of amino acids on both faces of Hcp1 leads us to propose a model whereby Hcp1 associates with proteins on both faces in order to facilitate the guided diffusion of a macromolecule through its inner pore.

### Hcp in Cystic Fibrosis Patients

Two observations indicate that hcp is expressed by P. aeruginosa in the lungs of some cystic fibrosis (CF) patients. First, the sputum of some CF patients contains hcp protein. Second, the sera of some CF patients contains antibodies that react with hcp. These data suggest that antibodies against hcp may be of therapeutic value to CF patients. Moreover, small molecule drugs that block secretion of hcp by P. aeruginosa may render the organism attenuated for virulence and thus allow patients to clear the organism from their lungs or perform better clinically.

### Discussion

We have found that the IAHP-I locus of P. aeruginosa is required for the secretion of Hcp1. Moreover, our data showing that proper subcellular localization of ClpB1* is IcmF1- and Hcp1-dependent provides a physical and functional linkage between components of the IAHP-I locus. These results, combined with the known association of IcmF to type IV secretion in L. pneumophila, and the requirement of AAA+ family ATPases for many secretory mechanisms, suggests that IAHP-I is likely to encode components of a novel secretion apparatus.

The finding that IAHP-I is specifically regulated by RetS, and that inactivating mutations of IAHP-I fail to be compensated for by orthologous genes of the other two loci suggests that these three pathways are not redundant and that their function is nonoverlapping. Perhaps this is to be expected given the broad spectrum of lifestyles represented by organisms with IAHP loci. Evidence for variability in IAHP function can also be garnered from its associated genetic constituents, which differ substantially between organisms and even between P. aeruginosa loci. As common components of signaling pathways, the serine-threonine kinase encoded by many, but not all IAHP loci, is one strong candidate for a mediator of such adaptive function. The IAHP-I and II loci of P. aeruginosa, both of which encode a serine-threonine kinase, also contain genes for proteins with strong homology to a serine-threonine phosphatase and an FHA domain-containing protein (FIG. 9). All of these genes are lacking from IAHP-III. The comigration of such a functionally-linked group of genes is indicative of their combined involvement in IAHP function.

The finding that P. aeruginosa IAHP-I belongs to the RetS regulon may provide insight critical for determining its function. Other well-characterized pathways repressed by RetS include those required for chronic infection and biofilm formation, such as the pel and psl operons, while those activated by RetS, such as type II and III secretion, and type IV pili, are major determinants of early stages of infection. Thus, IAHP-I would be expected to function late in infection, perhaps during biofilm formation in the cystic fibrosis lung. It is also of note that a screen for altered biofilm morphology in V. parahaemolyticus identified several independent tranposons insertion in hcp. These mutations caused cell aggregation and hastened the rate of biofilm detachment.

### Other Embodiments

All publications, patent applications, and patents mentioned in this specification are herein incorporated by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, microbiology or related fields are intended to be within the scope of the invention.

What is claimed is:

1. A method of treating or preventing a Gram-negative bacterial infection in a patient, said method comprising the steps of providing a humanized or human antibody or antibody fragment directed to a polypeptide selected from Hcp, VgrG, and Saf, and systemically administering the antibody to the patient, wherein the antibody treats or prevents Gram-negative bacterial infection.

2. The method of claim 1, wherein the Gram-negative bacterium is Pseudomonas aeruginosa, Salmonella enterica, Escherichia coli, Vibrio cholerae, Yersinia enterocolitica, Legionella pneumophilia, Enterobacter aerogenes, Proteus morganii, Klebsiella pneumoniae, Burkholderia cepacia, Burkholderia pseudomallei, Shigella flexneri, or Shigella dysenteriae.

3. The method of claim 2, wherein the Gram-negative bacterium is Pseudomonas aeruginosa.

4. The method of claim 3, wherein the patient is a burn patient, a surgical patient, a prosthesis recipient, a respiratory patient, a cancer patient, a cystic fibrosis patient, or an immunocompromised patient.

5. A method of treating or preventing Pseudomonas aeruginosa infection in a patient comprising the steps of providing a humanized or human antibody or antibody fragment directed to a polypeptide selected from Hcp, VgrG1, VgrG2, VgrG3, SafA, SafB, and SafC, and administering the antibody to the lungs of the patient.

6. The method of claim 5, wherein the patient is a burn patient, a surgical patient, a prosthesis recipient, a respiratory patient, a cancer patient, a cystic fibrosis patient, or an immunocompromised patient.

7. An antibody specific for a Gram-negative bacterium polypeptide selected from Hcp, VgrG, and Saf.

8. The antibody of claim 7, wherein the antibody is a monoclonal.

9. The antibody of claim 7, wherein the antibody or fragment thereof is humanized.

10. The antibody of claim 7, wherein the antibody or fragment thereof is human.

11. The antibody of any of claims 1-7, wherein the Gram-negative bacterium is Pseudomonas aeruginosa, Salmonella enterica, Escherichia coli, Vibrio cholerae, Yersinia enterocolitica, Legionella pneumophilia, Enterobacter aerogenes, Proteus morganii, Klebsiella pneumoniae, Burkholderia cepacia, Burkholderia pseudomallei, Shigella flexneri, or Shigella dysenteriae.

12. A pharmaceutical composition comprising the antibody or fragment of claim 7 and a pharmaceutically acceptable carrier.

13. The pharmaceutical composition of claim 12 in an amount effective for treating or preventing a Gram-negative bacterial infection in a patient.

14. The pharmaceutical composition of claim 12 in an amount effective for reducing the pathogenicity of a Gram-negative bacterial in a patient.

15. A method for treating or preventing a Gram-negative bacterial infection in a patient, said method comprising administering to the patient an effective amount of the composition of claim 12.

16. A method for reducing the pathogenicity of a Gram-negative bacterium in a patient, said method comprising administering to the patient an effective amount of the composition of claim 12.

17. The method of claim 15 or 16, wherein the Gram-negative bacterium is Pseudomonas aeruginosa, Salmonella enterica, Escherichia coli, Vibrio cholerae, Yersinia enterocolitica, Legionella pneumophilia, Enterobacter aerogenes, Proteus morganii, Klebsiella pneumoniae, Burkholderia cepacia, Burkholderia pseudomallei, Shigella flexneri, or Shigella dysenteriae.

18. The method of claim 17, wherein the Gram-negative bacterium is Pseudomonas aeruginosa.

19. The method of claim 18, wherein the patient is a burn patient, a surgical patient, a prosthesis recipient, a respiratory patient, a cancer patient, a cystic fibrosis patient, or an immunocompromised patient.

20. A method of inhibiting infection of a Gram-negative bacterium in a patient in need thereof, said method comprising administering to the patient an effective amount of an Hcp, VgrG, or Saf antigen.

21. The method of claim 20, wherein the antigen is a fragment capable of inducing an immune response.

22. The method of claim 20, wherein the patient is inoculated with a gene vaccine comprising DNA encoding the antigen.

23. The method of claim 22, wherein the DNA encodes a fragment capable of inducing an immune response.

24. The method of claim 20 wherein the patient is a human patient.

25. The method of any of claims 20-24, wherein the Gram-negative bacterium is Pseudomonas aeruginosa, Salmonella enterica, Escherichia coli, Vibrio cholerae, Yersinia enterocolitica, Legionella pneumophilia, Enterobacter aerogenes, Proteus morganii, Klebsiella pneumoniae, Burkholderia cepacia, Burkholderia pseudomallei, Shigella flexneri, or Shigella dysenteriae.

26. The method of claim 25, wherein the Gram-negative bacterium is Pseudomonas aeruginosa.

27. The method of claim 26, wherein the patient is a burn patient, a surgical patient, a prosthesis recipient, a respiratory patient, a cancer patient, a cystic fibrosis patient, or an immunocompromised patient.

28. A method of preventing or treating a Gram negative bacterial infection in a patient, said method comprising administering an effective amount of a compound that inhibits secretion or activity of Hcp or VgrG.

29. The method of claim 28, wherein the Gram-negative bacterium is Pseudomonas aeruginosa, Salmonella enterica, Escherichia coli, Vibrio cholerae, Yersinia enterocolitica, Legionella pneumophilia, Enterobacter aerogenes, Proteus morganii, Klebsiella pneumoniae, Burkholderia cepacia, Burkholderia pseudomallei, Shigella flexneri, or Shigella dysenteriae.

30. The method of claim 29, wherein the Gram-negative bacterium is Pseudomonas aeruginosa.

31. The method of claim 30, wherein the patient is a burn patient, a surgical patient, a prosthesis recipient, a respiratory patient, a cancer patient, a cystic fibrosis patient, or an immunocompromised patient.

32. An attenuated bacterial mutant, wherein said attenuated mutant contains a mutation of a gene of an IAHP locus.

33. The attenuated bacterial strain of claim 32, wherein said gene is a Pseudomonas aeruginosa gene selected from hcp1, hcp2, hcp3, vgrG1, vgrG2, vgrG3, icmF1, icmF2, icmF3, safA1, safA2, safA3, safB1, safB2, safB3, safC1, safC2, safC3, clpB1*, clpB2*, clpB3*, ppkA, pppA, fhaA, fhaB, stp1, and stk1.

34. The attenuated bacterial mutant of claim 32, wherein said mutation is insertional inactivation or a gene deletion.

35. The attenuated bacterial mutant of claim 32, wherein said mutant is a Gram-negative bacteria.

36. The attenuated bacterial mutant of claim 35, wherein said attenuated Gram-negative bacterial mutant is a Pseudomonas species.

37. The attenuated bacterial mutant of claim 36, wherein said Pseudomonas species is Pseudomonas aeruginosa.

38. The attenuated bacterial mutant of claim 32, wherein said attenuated Gram-negative bacterial mutant is Vibrio cholerae.

39. A method for identifying an antimicrobial drug, said method comprising: (a) contacting a candidate compound and a polypeptide encoded by a gene of an IAHP locus; and (b) comparing the biological activity of said polypeptide in the presence and absence of said candidate compound, wherein alteration of the biological activity of said polypeptide indicates that said candidate compound is an antimicrobial drug.

40. A method for identifying an antimicrobial drug, said method comprising: (a) contacting a candidate compound and a polypeptide encoded by a gene of an IAHP locus; and (b) detecting binding of said candidate compound and said polypeptide, wherein binding indicates that said candidate compound is an antimicrobial drug.

41. The method of claim 39 or 40, wherein said gene is a Pseudomonas aeruginosa gene selected from hcp1, hcp2, hcp3, vgrG1, vgrG2, vgrG3, icmF1, icmF2, icmF3, safA1, safA2, safA3, safB1, safB2, safB3, safC1, safC2, safC3, clpB1*, clpB2*, clpB3*, ppkA, pppA, fhaA, fhaB, stp1, and stk1.

42. A method for identifying an antimicrobial drug, said method comprising: (a) contacting a candidate compound and a Gram negative bacterium; and (b) detecting secretion of Hcp or VgrG, wherein a decrease in secretion, relative to secretion by said Gram negative bacterium not contacted with said candidate compound, indicates that said candidate compound is an antimicrobial drug.

43. The method of claim 42, wherein the Gram-negative bacterium is Pseudomonas aeruginosa, Salmonella enterica, Escherichia coli, Vibrio cholerae, Yersinia enterocolitica, Legionella pneumophilia, Enterobacter aerogenes, Proteus morganii, Klebsiella pneumoniae, Burkholderia cepacia, Burkholderia pseudomallei, Shigella flexneri, or Shigella dysenteriae.

44. The method of claim 43, wherein the Gram-negative bacterium is Pseudomonas aeruginosa.
